(19) United States
(12) Patent Application Publication
(10) Pub. No.: US 2019/0223745 A1
(43) Pub. Date: Jul. 25, 2019

(54) APPARATUS AND METHOD OF MEASURING FATIGUE

(71) Applicant: Maestro Co., Ltd., Seoul (KR)

(72) Inventors: Sang Ho LEE, Andong-si (KR); Jae Hwan SEOL, Seoul-si (KR); Joon Hyub LEE, Dong-gu (KR)

(73) Assignee: Maestro Co., Ltd., Seoul (KR)

(21) Appl. No.: 16/019,878

(22) Filed: Jun. 27, 2018

(30) Foreign Application Priority Data
Jan. 19, 2018 (KR) 10-2018-0007293

(51) Int. Cl. A61B 5/0476; A61B 5/16; A61B 5/00

(52) U.S. Cl. CPC A61B 5/0476; A61B 5/168; A61B 5/163; A61B 5/1103; A61B 5/6803; A61B 5/161; A61B 5/726

(57) ABSTRACT

An apparatus for measuring the fatigue of a user includes a first sensor configured to sense a factor causing the fatigue of a user; a second sensor configured to sense a brain wave signal of the user; and a processor configured to calculate a primary fatigue of the user based on the factor sensed by the first sensor, to calculate a secondary fatigue of the user based on the brain wave signal sensed by the second sensor, and to measure the fatigue of the user by using the primary fatigue and the secondary fatigue.

### CROSS-REFERENCE TO RELATED APPLICATION

This application claims the benefit of Korean Patent Application No. 10-2018-0007293, filed on Jan. 19, 2018, in the Korean Intellectual Property Office, the disclosure of which is incorporated herein in its entirety by reference.

### BACKGROUND

### 1. Field

One or more embodiments relate to an apparatus and method of measuring the fatigue of a user.

### 2. Description of the Related Art

The human brain is activated at its specific sites according to various activities. For example, when a person moves their arm, the area of the brain responsible for the motor center is activated, and the activation is measurable by using methods such as electroencephalography (EEG), functional magnetic resonance imaging (fMRI), magnetoencephalography (MEG), and near infrared spectroscopy (NIRs).

### SUMMARY

One or more embodiments include an apparatus and method of measuring the fatigue of a user.

Additional aspects will be set forth in part in the description which follows and, in part, will be apparent from the description, or may be learned by practice of the presented embodiments.

According to one or more embodiments, an apparatus for measuring the fatigue of a user includes: a first sensor configured to sense a factor causing the fatigue of a user; a second sensor configured to sense a brain wave signal of the user; and a processor configured to calculate a primary fatigue of the user based on the factor sensed by the first sensor, to calculate a secondary fatigue of the user based on the brain wave signal sensed by the second sensor, and to measure the fatigue of the user by using the primary fatigue and the secondary fatigue.

In one embodiment, the processor may quantitatively measure the fatigue of the user by setting a weight for each of the primary fatigue and the secondary fatigue, and performs computation using a weighted primary fatigue and a weighted secondary fatigue.

In one embodiment, the processor may adjust the weight by comparing the measured fatigue of the user to an actual fatigue of the user.

In one embodiment, when there are a plurality of factors, each being identical to the sensor sensed by the first sensor, the processor calculates the primary fatigue corresponding to values of the plurality of factors based on fuzzy logic.

In one embodiment, the first sensor may sense at least one selected from an amount of light exposed to eyes of the user, a neck posture of the user, an eye blink of the user, and a pupil movement of the user.

In one embodiment, the first sensor may include at least one selected from a light sensor, an acceleration/Gyro sensor, an electro-oculography (EOG) sensor, and an image sensor.

In one embodiment, the second sensor may measure a brain wave signal from a frontal lobe of the user.

In one embodiment, the second sensor may be an electroencephalogram (EEG) sensor.

In one embodiment, the processor may calculate the secondary fatigue by detecting a size of a specific frequency of the sensed brain wave signal through frequency analysis of the sensed brain wave signal.

In one embodiment, the apparatus may further include an output unit to provide content based on the measured fatigue of the user.

In one embodiment, the apparatus may include a wearable device.

In one embodiment, the apparatus may include a smart glass.

According to one or more embodiments, a method of measuring a fatigue of a user includes: sensing a user's fatigue-causing factor and a brain wave signal of a user; calculating a primary fatigue of the user based on the sensed user's fatigue-causing factor and a secondary fatigue of the user based on the sensed brain wave signal; and measuring the fatigue of the user by using the primary fatigue and the secondary fatigue.

According to one or more embodiments, a non-transitory computer-readable recording medium having recorded thereon a computer program, which, when executed by a computer, performs the method.

### BRIEF DESCRIPTION OF THE DRAWINGS

The present disclosure may be readily understood by reference to the following detailed description and the accompanying drawings, in which reference numerals refer to structural elements.

These and/or other aspects will become apparent and more readily appreciated from the following description of the embodiments, taken in conjunction with the accompanying drawings in which:

FIG. 1 illustrates a block diagram illustrating an apparatus for measuring the fatigue of a user according to an embodiment;

FIG. 2 illustrates an embodiment in which the apparatus measures a user's overall fatigue based on primary fatigue and secondary fatigue;

FIG. 3 illustrates an embodiment in which a processor calculates a user's primary fatigue based on fuzzy logic;

FIG. 4 illustrates an apparatus for measuring the fatigue of a user according to an embodiment;

FIG. 5 illustrates a method of measuring the fatigue of a user; and

FIG. 6 illustrates an apparatus for measuring the fatigue of a user according to an embodiment.

### DETAILED DESCRIPTION

With reference to the accompanying drawings, example embodiments will now be described in detail only for illustrative purpose. The following embodiments are merely intended to embody technical ideas and are not intended to limit the scope of disclosure. What is easily inferable by one of ordinary skill in the art in view of the detailed descriptions and embodiments is interpreted as being within the scope of the disclosure.

The terms “comprise” or “include” used herein should not be construed as necessarily including various components or operations described in the specification. For example, some of the components or operations may not be included, or additional components or operations will be further included. The terms “unit”, “module”, or the like refer to a unit that processes at least one function or operation, and the unit or module may be embodied by using hardware or software or a combination thereof.

The terms “first”, “second”, or the like used herein may refer to various components. However, these terms may be used to distinguish one component from another component or for illustrative purpose only.

Hereinafter, embodiments will be described in detail with reference to the drawings.

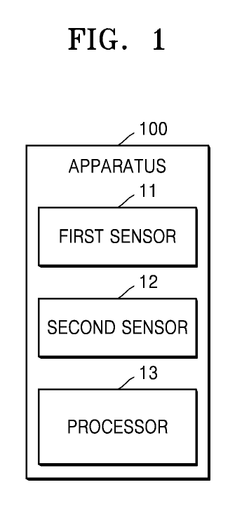

FIG. 1 illustrates a block diagram illustrating an apparatus 100 for measuring the fatigue of a user according to an embodiment.

The apparatus 100 for measuring the fatigue of a user (hereinafter, referred to as an apparatus for convenience.) includes a first sensor 11, a second sensor 12, and a processor 13. Regarding the apparatus 100 illustrated in FIG. 1, only components associated with the present embodiment are illustrated. Accordingly, one of ordinary skill in the art may understand that components related to the art other than the components illustrated in FIG. 1 may be additionally included.

In one embodiment, the apparatus 100 may be a wearable device, for example, smart glass.

The first sensor 11 may sense a factor that causes the fatigue of the user. Examples of the factor that causes the fatigue of the user are the amount of light exposure to the user's eyes, the posture of the user (for example, the posture of the user's neck), the number of blinks of the user's eyes, the movement of the user's eyes, and ocular electrical conductivity.

In one embodiment, the first sensor 11 may include a light sensor, an Acc/Gyro sensor, an electrooculogram (EOG) sensor, and an image sensor.

In one embodiment, the first sensor 11 may be a light sensor, and the first sensor 11 may sense the amount of light exposure to the eyes of a user. The first sensor 11 may be a light sensor of which a response characteristic with respect to a wavelength of light is similar to the response characteristic of human eyes. For example, the light sensor may be an ISL29101 product.

In one embodiment, the first sensor 11 may be an acceleration/Gyro sensor, and the first sensor 11 may sense the posture of a user. For example, the apparatus 100 may be a device that is wearable by a user. In this case, the first sensor 11 may sense the slope of the apparatus 100 to identify the posture of a user. For example, regarding an x-y-z coordinate system, the first sensor 11 may identify the posture of a user by sensing the acceleration components x, y, and z of the apparatus 100, and the angular velocity components x, y, and z of the apparatus 100.

In one embodiment, the first sensor 11 may be an image sensor, and in this case, the first sensor 11 may sense a user's eye blinking. Also, the first sensor 11 may detect the pupil of the user and track the movement of the pupil.

In one embodiment, the first sensor 11 may be an electrooculogram sensor (EOG), and may sense the ocular electrical conductivity of a user.

The processor 13 may control the operation of the apparatus 100, and process data and signals. The processor 13 may include at least one hardware unit. In one embodiment, the processor 13 may operate by one or more software modules generated by executing program code stored in a memory.

The processor 13 may calculate a primary fatigue of the user based on the user's fatigue-causing factor which has been sensed by the first sensor 11. In one embodiment, the processor 13 may calculate the user's primary fatigue by quantifying the user's fatigue-causing factor. For example, since the corresponding relationship or functional relationship between the user's fatigue-causing factor and the user's primary fatigue is set in advance, the processor 13 may, according to the corresponding relationship or the function relationship, calculate the primary fatigue of the user corresponding to the user's fatigue-causing factor.

In one embodiment, when there are many factors which cause the fatigue of the user, the processor 13 may calculate the user's primary fatigue by a computation using these factors. For example, since the corresponding relationship or functional relationship between the primary fatigue and each of the user's fatigue-causing factors is set in advance, the processor 13 may calculate the primary fatigue of the user corresponding to each of the user's fatigue-causing factors based on corresponding relationship or the function relationship. In one embodiment, the processor 13 may set a weight to each of the user's fatigue-causing factors, and may calculate the primary fatigue of the user based on a computation using the weighted factors.

The second sensor 12 may sense the brain wave signal of the user. In one embodiment, the second sensor 12 may sense the brain wave signal of the frontal lobe of the user. For example, the second sensor 12 may be an electroencephalogram (EEG) sensor.

The processor 13 may calculate a secondary fatigue of the user based on the user's brain wave signal sensed by the second sensor 12. The processor 13 may calculate the secondary fatigue of the user in such a way that the frequency of the brain wave signal of the user, measured by the second sensor 12, is analyzed to detect the size of a specific frequency of the brain wave signal.

The processor 13 may include an amplifier for amplifying the brain wave signal sensed by the second sensor 12, an analog processing unit that converts a brain wave signal to a digital signal, and a digital processing unit for processing digital signals. In one embodiment, the processor 13 may be embodied on a printed circuit board (PCB).

The processor 13 may measure the fatigue of a user based on the calculated primary fatigue and the calculated secondary fatigue. In other words, the processor 13 may quantitatively measure the overall fatigue of the user based on the calculated primary fatigue and the calculated secondary fatigue. In detail, the processor 13 may set a weight to each of the primary fatigue and the secondary fatigue. Then, the processor 13 may quantitatively measure the fatigue of the user by a computation using the weighted primary fatigue and the weighted secondary fatigue.

The processor 13 may variably adjust the weight that has been set for each of the primary fatigue and the secondary fatigue. In addition, the processor 13 may adjust the weight by comparing the measured fatigue of a user to the actual fatigue of the user. In detail, the processor 13 may obtain information about the actual fatigue experienced by the user, and may adjust the weight according to the degree of correlation between the actual fatigue and the measured fatigue. Therefore, the processor 13 sets the adjusted weight to the primary fatigue or the secondary fatigue, thereby accurately measuring the fatigue of a user.

As described above, the apparatus 100 measures the fatigue of a user by taking into account not only the user's brain wave signal but also user's fatigue-causing factors. Accordingly, the fatigue of a user may be accurately measured.

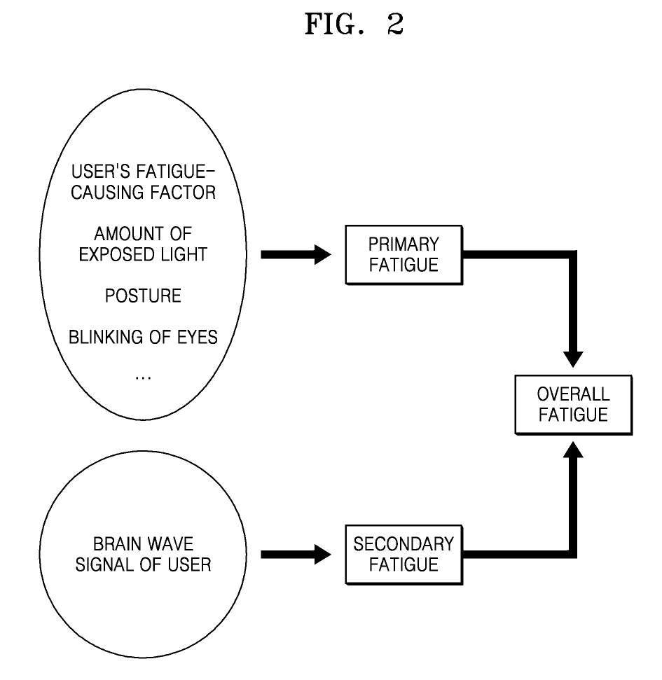

FIG. 2 illustrates an embodiment in which the apparatus 100 measures a user's overall fatigue based on the primary fatigue and the secondary fatigue.

The processor 13 may calculate the primary fatigue of a user based on a user's fatigue-causing factor.

In one embodiment, the first sensor 11 may measure the amount of light exposure to the eyes of a user. Then, the processor 13 may measure the load of the eyes according to the wavelength of light, and integrate the measured load of the eyes with respect to time to obtain the load of the light applied to the eyes of a user for a predetermined period of time. Accordingly, the processor 13 may calculate, as the primary fatigue, the load of light applied to the eyes of a user.

In one embodiment, the first sensor 11 may sense the posture of the neck of a user, and the processor 13 may calculate the stress applied to the neck of the user according to the sensed posture of the neck of the user as the primary fatigue of the user.

In one embodiment, the first sensor 11 may sense the user's brain wave signal, and the processor 13 may detect a particular frequency of the user's brain wave to determine whether the user is blinking. For example, the processor 13 may decompose the 0.1 Hz to 64 Hz signal, which is the brain wave signal of a user, into frequency-band signals by a multi-resolution signal decomposition technique of wavelet transform. For example, the 0.1 Hz to 64 Hz signal may be decomposed into four signal ranges: the 0 Hz to 8 Hz signal, the 8 Hz to 16 Hz signal, the 16 Hz to 32 Hz signal, and the 32 Hz to 64 Hz signal. In this regard, since the ocular electrical conduction caused by the movement of the eye muscles is measured with respect to the 0 Hz to 8 Hz signal, when the brain wave signal is in the 0 Hz to 8 Hz range, the processor 13 may identify that the user's eyes have blinked. The processor 13 then counts the number of blinks of the user's eyes and calculates the load that the user's eyes may receive as the user's primary fatigue.

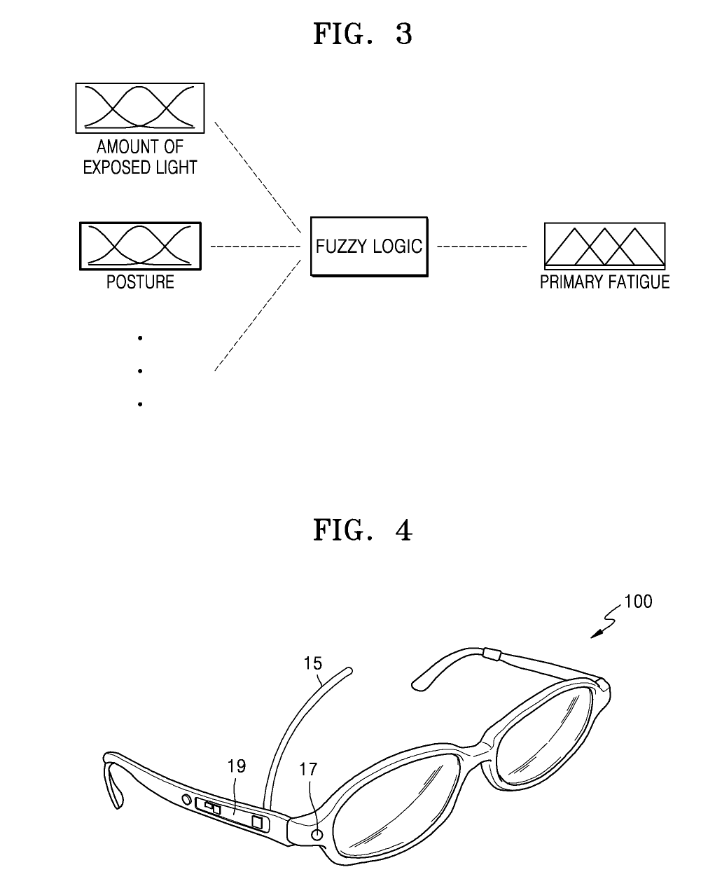

In one embodiment, when there are many user's fatigue-causing factors, the processor 13 may calculate the primary fatigue of the user based on fuzzy logic. Embodiments will be described below with reference to FIG. 3.

The processor 13 may calculate the user's secondary fatigue based on the user's brain wave signal. The processor 13 may calculate the secondary fatigue of a user in such a way that the frequency of the brain wave signal of a user, measured by the second sensor 12, is analyzed to detect the size of a specific frequency of the brain wave signal. In one embodiment, the processor 13 may perform a short-time Fourier transform (SFFT) on the brain wave signal of 0.1 Hz to 64 Hz to obtain the size of a signal according to frequency. In this regard, since the processor 13 may appropriately select a predetermined time interval, the stationary conditions of a FFT are satisfied and frequencies are obtained without frequency loss. For example, when measuring a brain wave signal of 0.1 Hz to 64 Hz, the processor 13 may perform an SFFT within a few seconds to obtain a frequency response without a large loss. The processor 13 then calculates the secondary fatigue of the user by calculating the ratio of the alpha wave of 8 Hz to 13 Hz representing mental relaxation to the beta wave of 13 Hz to 30 Hz representing mental concentration in the obtained response frequency. In addition, the processor 13 may calculate the secondary fatigue by analyzing the frequency of alpha and beta waves over time.

The processor 13 may measure the fatigue of a user based on the calculated primary fatigue and secondary fatigue. Specifically, the processor 13 may quantitatively measure the fatigue of a user by setting a weight to each of the primary fatigue and the secondary fatigue, and performing a computation using the weighted primary fatigue and the weighted secondary fatigue. For example, the processor 13 may calculate the total fatigue of a user by using Equation 1.

Fatigue of user=weight of primary fatigue (w1)×primary fatigue+weight of secondary fatigue (w2)×secondary fatigue [Equation 1]

For example, when the primary fatigue is 80, the secondary fatigue is 60, the weight of the primary fatigue is 0.3, and the weight of the secondary fatigue is 0.7, the total fatigue of a user, calculated by the processor 13 using Equation 1, is 66. In addition, the processor 13 may re-adjust the weights by comparing the measured fatigue of a user to the actual fatigue of the user.

FIG. 3 illustrates an embodiment in which the processor 13 calculates a user's primary fatigue based on fuzzy logic.

The processor 13 may calculate the primary fatigue of a user corresponding to the value of each of user's fatigue-causing factors, based on fuzzy logic. In detail, the processor 13 may calculate the primary fatigue of the user corresponding to the values of a plurality of factors sensed by the first sensor 11, according to a plurality of factors and the primary fatigue set as an input and output of fuzzy logic.

In one embodiment, the processor 13 may fuzzify each of the values of the factors that cause fatigue of the user and the value of the primary fatigue as an input and output of the fuzzy logic. For example, as an input to fuzzy logic, the processor 13 may fuzzify the value of ‘light exposure amount’ into 3 ranges: low, medium, and high, and the value of ‘posture’ into 3 ranges: bad, moderate, and good. Also, as the output of fuzzy logic, the processor 13 may fuzzify the value of the primary fatigue into five ranges: very bad, bad, moderate, good, and very good. Then, the processor 13 may set the relationship between the input and the output of the fuzzy logic according to a predetermined fuzzy rule. For example, when each of the three factors, which are inputs to the fuzzy logic, is divided into three ranges, the combination of inputs is as follows: 3×3×3=27 When the primary fatigue, which is the output of the fuzzy logic, is divided into 5 ranges, the relationship between the input of 27 cases and the output of 5 cases may be set according to a certain purge rule.

Thus, based on the relationship between the input and the output set according to a predetermined fuzzy rule, the processor 13 may calculate the primary fatigue of the user corresponding to the values of the plurality of factors measured by the first sensor 11.

FIG. 4 illustrates an example of the apparatus 100 for measuring fatigue of a user.

As illustrated in FIG. 4, the apparatus 100 may be embodied in the form of a smart glass.

The apparatus 100 may include an EEG sensor 15, a light sensor 17, and a printed circuit board (PCB) 19. The apparatus 100 of FIG. 4 is the same as described in connection with the apparatus 100 of FIG. 1, and only the difference between the apparatus 100 of FIG. 4 and the apparatus 100 of FIG. 1 will now be described.

The apparatus 100 may include the light sensor 17 as the first sensor 11. As illustrated in FIG. 4, the light sensor 17 is attached to a front portion of the apparatus 100, and thus, may accurately sense the amount of light exposure to the eyes of the user.

The apparatus 100 may include the EEG sensor 15 as the second sensor 12. The EEG sensor 15 may be embodied in the form of a bar that is configured to be in close contact with the forehead of the user. For example, the EEG sensor 15 may be embodied as a tension bar that continuously applies a pressing force to a portion of the user's head. Thus, the EEG sensor 15 may sense the brain wave signal from the frontal lobe of the user. In one embodiment, the apparatus 100 may further include a ground (GND) terminal or a reference terminal in association with the operation of the EEG sensor 15.

The apparatus 100 may include the PCB 19, and the PCB 19 may include a processor 13 and an micro electro mechanical systems (MEMS) sensor for measuring gravitational acceleration and angular acceleration.

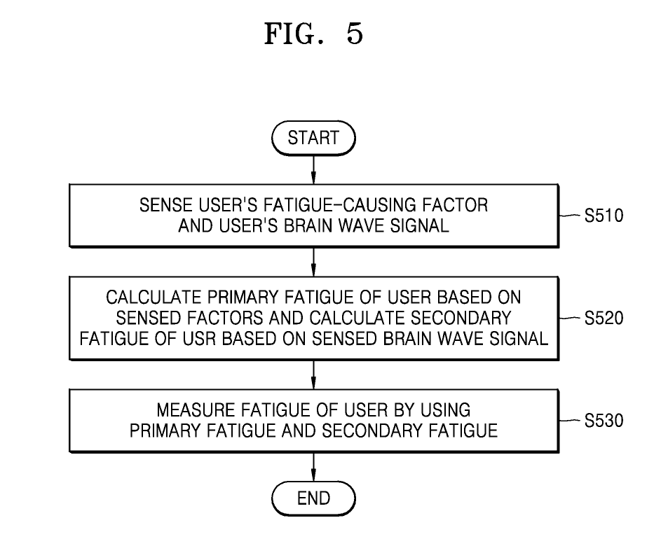

FIG. 5 illustrates a method of measuring the fatigue of a user.

The method shown in FIG. 5 may be performed by each component of the apparatus 100 of FIGS. 1 to 4. Accordingly, redundant description thereof will be omitted.

In operation S510, the apparatus 100 may sense a user's fatigue-causing factor and sense a user's brain wave signal.

The apparatus 100 may sense the amount of light exposure to the user's eyes by using a light sensor. Also, the apparatus 100 may sense the posture of a user by using an acceleration/Gyro sensor. In addition, the apparatus 100 may sense an eye blink of a user or a pupil movement of a user by using an image sensor or an EOG sensor.

The apparatus 100 may sense the user's brain wave signal by using the EEG sensor. For example, the apparatus 100 may sense the brain wave signal from the user's frontal lobe.

In operation S520, the apparatus 100 may calculate the primary fatigue of the user based on a sensed factor and may calculate the secondary fatigue of the user based on the sensed brain wave signal.

The apparatus 100 may calculate the user's primary fatigue by quantifying the user's fatigue-causing factor. In addition, the apparatus 100 may calculate the secondary fatigue of a user by detecting the size of a specific frequency of a brain wave signal through the analysis of a frequency of a user's brain wave signal.

In operation S530, the apparatus 100 may measure the fatigue of a user by using the primary fatigue and the secondary fatigue. The apparatus 100 may set a weight to each of the primary fatigue and the secondary fatigue. Then, the apparatus 100 may quantitatively measure the fatigue of a user by a calculation using the weighted primary fatigue and the secondary fatigue.

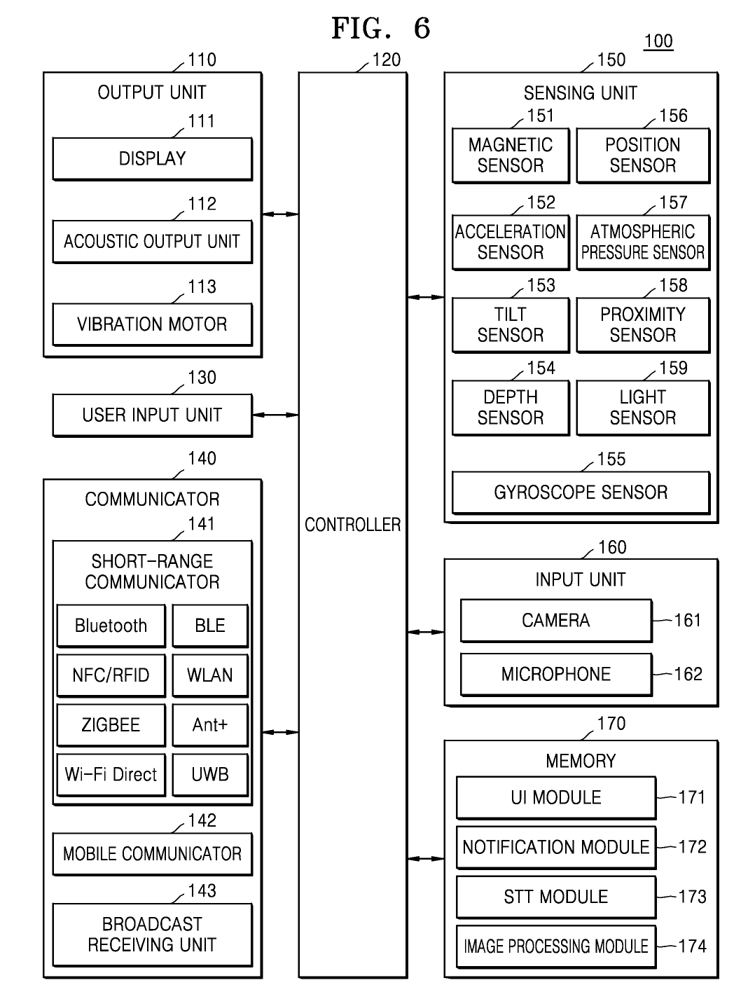

FIG. 6 illustrates another example of the apparatus 100 for measuring the fatigue of a user.

The apparatus 100 may include an output unit 110, a controller 120, a user input unit 130, a communicator 140, a sensing unit 150, an audio/video (NV) input unit 160, and a memory 170. In addition, the processor 13 of FIG. 1 may correspond to the controller 120 of FIG. 6, and the first sensor 11 or the second sensor 12 of FIG. 1 may correspond to the sensing unit 150 of FIG. 6. The sensing unit 150 may also be referred to as a sensor. The apparatus 100 of FIG. 6 is the same as described in connection with the apparatus 100 of FIGS. 1 to 4, and only the difference between the apparatus 100 of FIG. 4 and the apparatus 100 of FIGS. 1 to 4 will now be described.

The output unit 110 outputs an audio signal, a video signal, or a vibration signal, and may include a display unit 111, an acoustic output unit 112, a vibration motor 113, and the like.

The display unit 111 may display information processed by the apparatus 100. For example, when the fatigue of a user, measured by the controller 120, is high, the display unit 111 may display content (notification message information). In this regard, the display unit 111 may display content in the form of augmented reality (AR), mixed reality (MR), or virtual reality (VR).

Meanwhile, when the display unit 111 and a touch pad are configured to have a layer structure constituting a touch screen, the display unit 111 may be used as an input device and an output device. The display unit 111 may include at least one selected from a liquid crystal display, a thin film transistor-liquid crystal display, an organic light-emitting diode display, a flexible display, a 3D display, and an electrophoretic display. In one or more embodiments, the apparatus 100 may include two or more display units, each being the display unit 111.

The acoustic output unit 112 may output audio data received from the communicator 140 or stored in the memory 170. Also, the acoustic output unit 112 outputs an acoustic signal related to the functions (for example, incoming call sound, message reception sound, alarm sound) performed in the apparatus 100. The acoustic output unit 112 may include a speaker, a buzzer, and the like.

The vibration motor 113 may output a vibration signal. For example, the vibration motor 113 may output a vibration signal corresponding to an output of audio data or video data (e.g., incoming call sound, message reception sound, etc.). In addition, the vibration motor 113 may output a vibration signal when a touch is input to a touch screen.

The output unit 110 may provide content based on the measured fatigue of a user. For example, when the fatigue of a user, measured by controller 120, is high, the output unit 110 may provide content to reduce the fatigue of a user.

The controller 120 controls the overall operation of the apparatus 100. For example, the controller 120 may overall control the output unit 110, the user input unit 130, the communicator 140, the sensing unit 150, the A/V input unit 160, etc. by executing programs stored in the memory 170.

The controller 120 may determine whether a user is wearing a wearable glass based on a signal output by at least one sensor included in the sensing unit 150. When it is determined that the user is wearing the wearable glass, the fatigue of a user is measured.

The user input unit 130 includes a means by which the user inputs data for controlling the apparatus 100. For example, the user input unit 130 may be a keypad, a dome switch, a touch pad (a contact-type capacitive method, a pressure type resistive membrane type method, an infrared detection system, a surface ultrasonic wave conduction method, an integral tension measurement method, a piezoelectric effect method, etc.), a jog wheel, a jog switch, and the like, but is not limited thereto.

The communicator 140 may include one or more components enabling communication between the apparatus 100 and a mobile terminal, the apparatus 100 and a server, and between the apparatus 100 and an external wearable device. For example, the communicator 140 may include a short-range wireless communicator 141, a mobile communicator 142, and a broadcast receiving unit 143.

The short-range wireless communicator 141 may include a Bluetooth communicator, a Bluetooth low energy (BLE) communicator, a near field communicator, a WLAN (Wi-Fi) communicator, a ZigBee communicator, IrDA, an infrared data association (WDF) communication unit, a Wi-Fi Direct (WFD) communication unit, an ultra wideband (UWB) communicator, and an Ant⁺ communicator, but is not limited thereto.

The mobile communicator 142 may transmit and receive wireless signals to or from at least one of a base station, an external terminal, and a server on a mobile communication network. Here, the wireless signal may include various types of data depending on transmission and reception of a voice call signal, a video call signal, or a text/multimedia message.

The broadcast receiving unit 143 receives broadcast signals and/or broadcast-related information from outside through a broadcast channel. The broadcast channel may include a satellite channel and a terrestrial channel. According to one or more embodiments, the apparatus 100 may not include the broadcast receiving unit 143.

The sensing unit 150 may sense the state of the apparatus 100, the state of the surroundings of the apparatus 100, the state of a user wearing the apparatus 100, the movement of a user, and transmit the sensed information to the controller 120. For example, the sensing unit 150 senses the movement of the user and outputs a signal related to the movement of the user to the controller 120, wherein the signal may be an electrical signal.

The sensing unit 150 may include at least one selected from a magnetic sensor 151, an acceleration sensor 152, a tilt sensor 153, a depth sensor 154, a Gyroscope sensor 155, a position sensor [e.g., global positioning system (GPS)] 156, an atmospheric pressure sensor 157, a proximity sensor 158, and a light sensor 159, but is not limited thereto. The sensing unit 150 may include a temperature sensor, an illumination sensor, a pressure sensor, an iris recognition sensor, and the like. The function of each of these sensors may be intuitively deduced from their names by those skilled in the art, so a detailed description will be omitted.

The A/V input unit 160 is used for inputting an audio signal or a video signal, and may include a camera (image sensor) 161 and a microphone 162. The camera (image sensor) 161 may obtain an image frame such as a still image or a moving image in a video communication mode or a shooting mode. The image captured by the camera (image sensor) 161 may be processed through the controller 120 or a separate image processor (not shown).

An image frame processed in the camera (image sensor) 161 may be stored in the memory 170 or may be transmitted to the outside via the communicator 140. In one or more embodiments, the apparatus 100 may include two or more cameras (image sensor), each being identical to the camera (image sensor) 161.

The microphone 162 receives an external acoustic signal and processes the external acoustic signal into electrical voice data. For example, the microphone 162 may receive acoustic signals from an external device or a speaking person. The microphone 162 may use various noise reduction algorithms to remove noises generated during when the external acoustic signal is received.

The memory 170 may store a program for processing and controlling the controller 120, and may store input/output data (e.g., a list of unreleased content, a list of output content, a captured image, biometrics, schedule information about the user, life pattern information about the user, and the like).

The memory 170 may include at least one storage media selected from a flash memory type memory, a hard disk type memory, a multimedia card micro type memory, a card type memory (for example, an SD or XD memory, etc.), random access memory (RAM), static random access memory (SRAM), read only memory (ROM), electrically erasable programmable read-only memory (EEPROM), programmable read-only memory (PROM), a magnetic memory, a magnetic disc, and an optical disc. In addition, the apparatus 100 may operate a web storage or a cloud server that performs a storage function of the memory 170 on the Internet.

Programs stored in the memory 170 may be classified into a plurality of modules according to their functions, for example, into a user interface (UI) module 171, a notification module 172, a speak-to-text (STT) module 173, an image processing module 174, and the like.

The UI module 171 may provide a specialized UI, a graphical user interface (GUI), and the like, which are associated with with the apparatus 100 for each application. The notification module 172 may generate a signal for notifying the occurrence of an event of the apparatus 100. The notification module 172 may output a notification signal in the form of a video signal through the display unit 111, a notification signal in the form of an audio signal through the acoustic output unit 112, or a notification signal in the form of a vibration signal through the vibration motor 113.

The STT module 173 may convert a voice included in multimedia content into a text to generate a transcript corresponding to the multimedia content.

The image processing module 174 may obtain information about an object, information about an edge, information about the atmosphere, information about color, and the like in the captured image by analysis of the captured image.

A server or a device according to the above embodiments may include a processor, a memory for storing and executing program data, a permanent storage such as a disk drive, a communication port for communicating with an external apparatus, a user interface apparatus such as a touch panel, a key, or a button, etc. Methods implemented with software modules or algorithms may be stored as computer readable code or program instructions executable on a processor on a computer-readable recording medium. Examples of the computer-readable recording medium include magnetic storage media (e.g., ROM, RAM, floppy disks, hard disks, etc.), and optical recording media (e.g., CD-ROMs, or digital versatile discs (DVDs)). The computer-readable recording medium can also be distributed over network-coupled computer systems so that the computer-readable code is stored and executed in a distributive manner. This media can be read by the computer, stored in the memory, and executed by the processor.

The present embodiments may be described in terms of functional block components and various processing steps. Such functional blocks may be realized by any number of hardware and/or software components configured to perform the specified functions. For example, embodiments may employ various integrated circuit (IC) components, such as memory elements, processing elements, logic elements, look-up tables, and the like, which may carry out a variety of functions under the control of one or more microprocessors or other control devices. Similarly, where the elements are implemented using software programming or software elements, the present embodiments may be implemented with any programming or scripting language such as C, C++, Java, assembler language, or the like, with the various algorithms being implemented with any combination of data structures, objects, processes, routines or other programming elements. The functional blocks may be implemented in algorithms that are executed on one or more processors. Furthermore, the present embodiment described herein could employ any number of techniques of the related art for electronics configuration, signal processing and/or control, data processing and the like. The terms “mechanism”, “element”, “means”, and “configuration” are used broadly and are not limited to mechanical or physical embodiments, but can include software routines in conjunction with processors, etc.

The particular implementations shown and described herein are illustrative examples and are not intended to otherwise limit the scope of the present invention in any way. For the sake of brevity, electronics of the related art, control systems, software development and other functional aspects of the systems may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a device used in the art.

The use of the terms “a”, “an”, and “the” and similar referents in the context of describing the present invention (especially in the context of the following claims) are to be construed to cover both the singular and the plural. Furthermore, recitation of ranges of values herein are merely intended to serve as each separate value falling within the range, unless otherwise indicated herein, and each separate value is incorporated into the specification as if it were individually recited herein. The steps of all methods described herein can be performed in any suitable order unless otherwise indicated herein or otherwise clearly contradicted by context. The order of the steps of all methods is not limited thereto. The use of any and all examples, or exemplary language (e.g., “such as”) provided herein, is intended merely to better illuminate the inventive concept and does not pose a limitation on the scope of the inventive concept unless otherwise claimed. It will be apparent to one of ordinary skill in the art that numerous modifications and adaptations may be made according to design conditions or factors without departing from the accompanying claims or their equivalents.

According to embodiments of the present disclosure, the fatigue of a user is measured by taking into account not only the user's brain wave signal but also user's fatigue-causing factors. Accordingly, the fatigue of a user may be accurately measured. Also, by comparing the measured fatigue of a user to the actual fatigue of a user, the weight set to each of the primary fatigue and the secondary fatigue is controlled, thereby enabling accurate measurement of the fatigue of a user.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. An apparatus for measuring the fatigue of a user, the apparatus comprising:
a first sensor configured to sense a factor causing the fatigue of a user;
a second sensor configured to sense a brain wave signal of the user; and
a processor configured to calculate a primary fatigue of the user based on the factor sensed by the first sensor, to calculate a secondary fatigue of the user based on the brain wave signal sensed by the second sensor, and to measure the fatigue of the user by using the primary fatigue and the secondary fatigue.

2. The apparatus of claim 1, wherein
the processor quantitatively measures the fatigue of the user by setting a weight to each of the primary fatigue and the secondary fatigue, and performs a computation using a weighted primary fatigue and a weighted secondary fatigue.

3. The apparatus of claim 2, wherein
the processor adjusts the weight by comparing the measured fatigue of the user to an actual fatigue of the user.

4. The apparatus of claim 1, wherein,
when there are a plurality of factors, each being identical to the factor sensed by the first sensor,
the processor calculates the primary fatigue corresponding to values of the plurality of factors based on fuzzy logic.

5. The apparatus of claim 1, wherein
the first sensor senses at least one selected from an amount of light exposure to the eyes of the user, a neck posture of the user, an eye blink of the user, and a pupil movement of the user.

6. The apparatus of claim 1, wherein
the first sensor comprises at least one selected from a light sensor, an acceleration/Gyro sensor, an electro-oculography (EOG) sensor, and an image sensor.

7. The apparatus of claim 1, wherein
the second sensor measures a brain wave signal from a frontal lobe of the user.

8. The apparatus of claim 1, wherein
the second sensor comprises an electroencephalogram (EEG) sensor.

9. The apparatus of claim 1, wherein
the processor calculates the secondary fatigue by detecting a size of a specific frequency of the sensed brain wave signal through frequency analysis of the sensed brain wave signal.

10. The apparatus of claim 1, further comprising
an output unit to provide content based on the measured fatigue of the user.

11. The apparatus of claim 1, wherein
the apparatus comprises a wearable device.

12. The apparatus of claim 1, wherein
the apparatus comprises a smart glass.

13. A method of measuring a fatigue of a user, the method comprising:
sensing a user's fatigue-causing factor and a brain wave signal of the user;
calculating a primary fatigue of the user based on the sensed user's fatigue-causing factor and a secondary fatigue of the user based on the sensed brain wave signal; and
measuring the fatigue of the user by using the primary fatigue and the secondary fatigue.

14. A non-transitory computer-readable recording medium having recorded thereon a computer program, which, when executed by a computer, performs the method of claim 13.
